(19) United States
(12) Patent Application Publication
(10) Pub. No.: US 2004/0248306 A1
(43) Pub. Date: Dec. 9, 2004

(54) Microfluidic water analytical device

(76) Inventors: Juan J. Hernandez, San Diego, CA (US); Alicia Chen, Corvallis, OR (US); Dennis W. Tom, Corvallis, OR (US); Dustin W. Blair, San Diego, CA (US)

Correspondence Address: HEWLETT-PACKARD COMPANY, Intellectual Property Administration, P.O. Box 272400, Fort Collins, CO, 80527-2400, US

(21) Appl. No.: 10/457,993

(22) Filed: Jun. 9, 2003

(51) Int. Cl. G01N 33/18

(52) U.S. Cl. 436/39; 422/58

(57) ABSTRACT

The illustrated invention defines an analytical chip comprising an optically transparent substrate such as glass or silicon that defines a fluid inlet port and at least one fluid carrying channel communicating with the inlet. At least one reaction chamber fluidly communicates with the channel and an air management chamber is in fluid communication with the reaction chamber to facilitate capillary flow of fluid into the reaction chamber.

### TECHNICAL FIELD

This invention relates to apparatus for analyzing fluid samples, especially water, and more specifically, to an analysis chip having microfluidic sample handling channels and associated reaction chambers for field-testing water samples.

### BACKGROUND OF THE INVENTION

Analytical testing of water samples plays an important role in determining water quality in innumerable settings, from large municipal water providers and industrial users to homeowners with wells. There are hundreds of water quality parameters that may be tested. Some of the more common analytical tests that are routinely performed as a measure of water quality include, temperature, pH, chlorine, sulfates, phosphates, hardness, alkalinity, nitrates, dissolved oxygen, turbidity, total organic carbon, and biological oxygen demand.

An entire industry has developed to supply analytical instruments and testing kits that are specifically for use in performing water analysis. These instruments include everything from sophisticated and expensive laboratory instrumentation on one end of the spectrum, to relatively inexpensive portable test kits and test strips on the other end. The type of instrumentation and testing that is done depends of course on the particular need. In some cases technicians are able to rely upon sophisticated laboratory instruments to run both routine tests and more sophisticated analyses. Such laboratory instruments are well suited for use in the controlled conditions found in an analytical lab. However, in many situations it is necessary to run analytical tests on water samples in the field in order to obtain quick analytical results as a measure of water quality. Traditional laboratory instruments are not designed for use in the field, and as a result, there is a need for specialized analytical equipment designed for use in field-testing of water samples. However, analytical water analysis kits and equipment that are designed to withstand the rigors of field use often do not provide results that have the desired accuracy or precision.

Field-testing of water to determine the amount or presence of certain compounds and chemicals in water, or other physical and chemical attributes of a water sample is of great practical importance. For example, municipal water systems must routinely test the water to ensure that it is in compliance with regulations, and is suitable for consumption. Municipal water systems therefore perform water analyses on a continuous basis, both in the field and in the lab. Likewise, industries that use process water must test the wastewater to ensure that it meets regulatory standards. Moreover, many industries that use large volumes of process water must monitor the quality of discharged water, such as the biochemical oxygen demand (BOD), on an ongoing basis to that effluent complies with appropriate standards. It is thus important for such industries to have accurate data regarding the condition of wastewater.

While there are numerous ongoing advances being made in analytical chemistry that are providing promising techniques and apparatus for use in field-testing of water, it can be appreciated that a need exists for apparatus capable of rapidly and accurately running analyses of water samples. There is an especially significant and ongoing need for apparatus and methods that allow for running a variety of chemical and physical analyses of a water sample in the field. Such instruments desirably will include the ability to run multiple tests for a variety of sample attributes, be simple to operate and use so that the level of operator training is reduced, and will have a small size so that they can be easily transported to the field for on-site use.

Apparatus and methods addressing these needs are described in detail below. Advantages and features of the illustrated invention will become clear upon review of the following specification and drawings.

### SUMMARY

The illustrated embodiment is an analysis chip comprising a member defining a fluid inlet, at least one fluid carrying channel fluidly connected to the inlet, and at least one reaction chamber fluidly connected to the at least one fluid carrying channel. An air management chamber is connected to the reaction chamber.

### BRIEF DESCRIPTION OF THE DRAWINGS

FIG. 1 is a perspective, schematic view of a water analysis chip in accordance with one illustrated embodiment of the invention.

FIG. 2 is a top plan view of the water analysis chip illustrated in FIG. 1, showing in phantom lines the microfluidic channels, reaction chambers and other structures contained in the chip.

FIG. 3 is perspective view of the upper layer of the water analysis chip shown in FIG. 1 with layer inverted to reveal the fluid ports, reaction chambers and microfluidic channeling.

FIG. 4 is a cross sectional view taken along the line 4-4 of FIG. 2 and illustrating the electrical interconnect components used for certain sample analyses.

FIG. 5 is cross sectional view taken along the line 5-5 of FIG. 2 and illustrating three separate reaction chambers.

FIG. 6 is a schematic view of the water analysis chip shown in FIG. 1 and associated analytical instrumentation used to gather, compile and store analytical data from the chip.

FIG. 7 is a photomicrograph of an alternative embodiment of the illustrated invention, showing a four-terminal electrical interconnect in one reaction chamber.

FIG. 8 is a top plan view of the upper board of yet another water analysis chip in accordance with the illustrated embodiment of the invention.

FIG. 9 is a flow diagram illustrating operational steps used to analyze a water sample with the illustrated water analysis chip.

### DETAILED DESCRIPTION OF ILLUSTRATED EMBODIMENTS

The illustrated invention provides an integrated, self-contained optically transparent apparatus for acquiring a fluid sample, and routing the sample through microfluidic channels into various reaction chambers by passive capillary action. A variety of qualitative and/or quantitative analyses of the sample may be performed. While the inventive apparatus may be used in numerous situations, it is especially useful for field analysis of a water sample where more traditional sample collection and analytical instruments are difficult or impossible to use. Moreover, although the invention is described herein primarily with respect to its use as an analytical device for use in sampling and analyzing water, it may just as well be used to analyze other fluids.

The illustrated invention comprises a microfluidic chip apparatus that in one embodiment incorporates one or more reaction chambers where the fluid sample—typically water—is tested. Three different types of reaction chambers are illustrated. The first type of reaction chamber facilitates chemical-based tests of a water sample. These reaction chambers typically have various analytical reagents and or dyes deposited therein that react in known ways with water. Each chip may include a plurality of these chemical reaction chambers, and each of these may contain reagents that test for a different property. Each chip may thus be customized so that any number of different chemical tests may be run with a single chip. The second type of reaction chamber is configured to facilitate electrical analyses of a water sample and includes circuitry that allows various electrical tests to be run. Plural electrical reaction chambers may be included on a single chip, so different electrical tests may be run with a single chip. The third type of reaction chamber is a blank chamber that utilizes neither analytical reagents nor electrical circuitry, and is intended to facilitate evaluation of sample contained in the chamber for properties such as turbidity and color. This third type of chamber is referred to herein as an optical chamber.

The water analysis chip described herein is used with an analytical instrument designed especially for use with the chip. The analytical instrument is designed to detect calorimetric changes that occur in water samples in the chemical reaction chambers, optical characteristics and electrical properties of water samples in the electrical reaction chambers, and based on the detected changes, provide an output useful as an analytical measure of a specific tested parameter. The instrument may be connected to a microprocessor such as a personal digital assistant or laptop computer for rapid collection and storage of data acquired in the field. The analytical instrument is described generally herein to facilitate understanding of the invention.

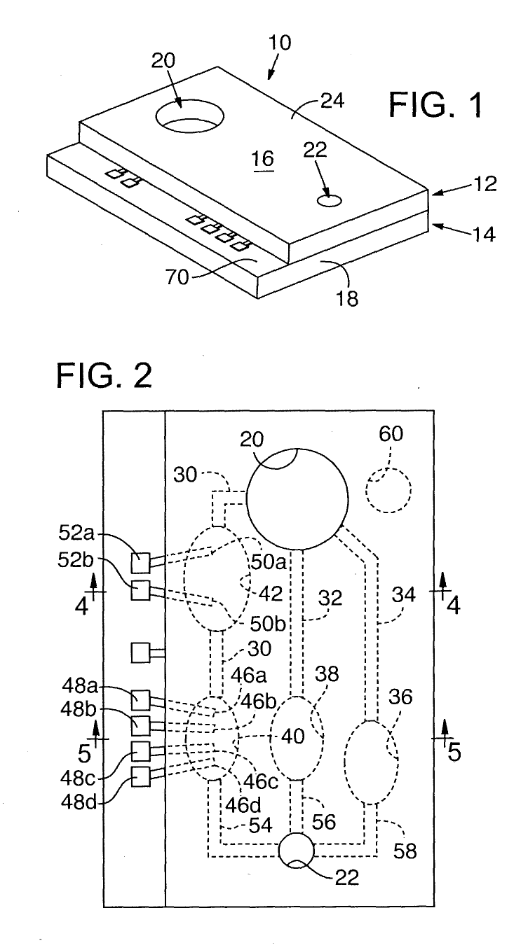

FIG. 1 is a schematic reproduction in a graphic form of a single water analysis chip 10 configured for the performance of water sample acquisition and analysis in accordance with one aspect of the illustrated invention. It will be appreciated that the water analysis chip 10 illustrated in FIG. 1 is shown in a highly schematic fashion to provide detailed information about the structure and operation of the chip.

Chip 10 is depicted in perspective form in FIG. 1 and comprises a composite substrate member defined by an upper board 12 and a lower board 14. As described below, each board 12 and 14 is separately fabricated. The two boards 12 and 14 may be fabricated from a variety of materials, including glasses, silicon materials and even plastics.

Upper board 12 is an orifice-containing plate that defines various fluid ports, channels and reaction chambers, and thus defines the water analysis chip 16. The lower board 14 contains the electrical interconnects and bond pads that interface the chip 10 with the analytical instrument 80 described below, and thus defines the electrical chip 18.

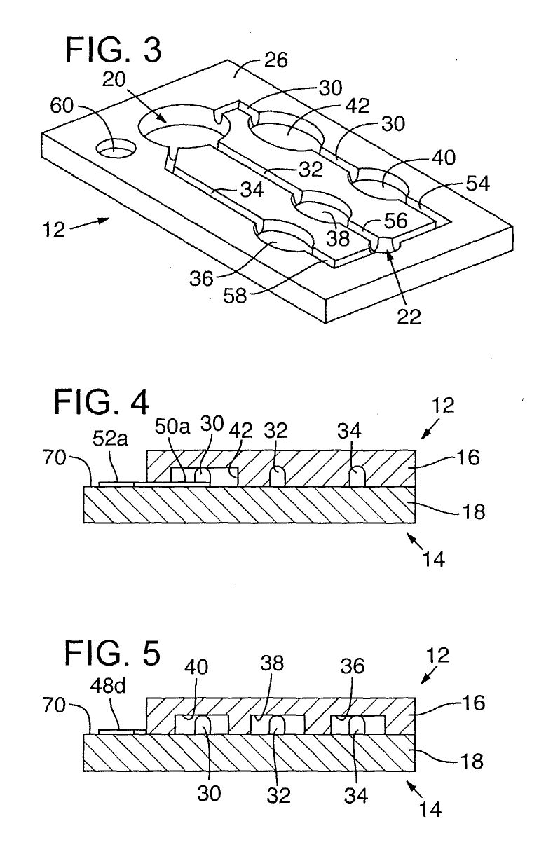

With reference now to FIGS. 1, 2 and 3, upper board 12 has a fluid inlet port 20 and an air management port 22, each of which defines an opening through the upper surface 24 of upper board 12 that fluidly communicates with the fluid-carrying microfluidic channels formed in the lower surface 26 (see FIG. 3) of the upper board. A plurality of fluid-carrying channels, labeled with reference numbers 30, 32 and 34 are formed (in the manner described below) in the lower surface 26 of upper board 12. Each of these channels 30, 32 and 34 defines a pathway that fluidly communicates at a first end with fluid inlet port 20 and at a second end with air management port 22. Plural reaction chambers are interposed in the fluid-carrying microfluidic channels, and in FIGS. 2 through 5 the reaction chambers are labeled with reference numbers 36, 38, 40 and 42. For purposes of illustration, reaction chamber 36 is a chemical type reaction chamber because, as described below, chemical reactions are carried out in this reaction chamber. It will be appreciated that an actual chip 10 will include many chemical reaction chambers such as chamber 36, and each of the chemical reaction chambers 36 may be configured for testing a water sample for a different attribute or parameter, such as the presence or concentration of a given chemical, etc. Chamber 38 is an optical chamber that, as noted above, is not associated with any reagents or dyes. Again, an actual chip 10 will include plural optical chambers such as chamber 38. Reaction chambers 40 and 42 are electrical type reaction chambers because they are configured for testing electrical properties of water contained in the chambers.

It may be seen from FIG. 2 that each of the fluid-carrying microfluidic channels 30, 32 and 34 defines a fluid pathway that fluidly communicates between fluid entry port 20 and air management port 22. As detailed below, air management port 22 is provided to control and manage sample fluid movement through the fluid-carrying channels and into the reaction chambers by facilitating capillary fluid flow. The term “passive capillarity” is used at times herein because the capillary fluid flow is not induced with any active mechanisms. The portions of the fluid-carrying channels between the reaction chambers and the air management port are at times referred to as air management channels 54, 56 and 58. It should be noted, however, that a direct fluid pathway from the fluid inlet port 20 through the reaction chambers and to the air management port 22 is not required. One example illustrating this later structure is described in reference to FIG. 7. Moreover, the air management port 22 need not be open to atmosphere as illustrated in FIG. 1, and instead may be a chamber that defines an air management port that is not open to atmosphere.

A reference cell 60 is formed in the lower surface 26 of upper board 12 but is not fluidly connected to any other channel or reaction chamber, and does not communicate with the upper surface 24 of upper board 12. It will be appreciated that the number of microfluidic channels and reaction chambers, and the number of reaction chambers interposed in any given channel, may be varied from the schematic illustration shown in the figures.

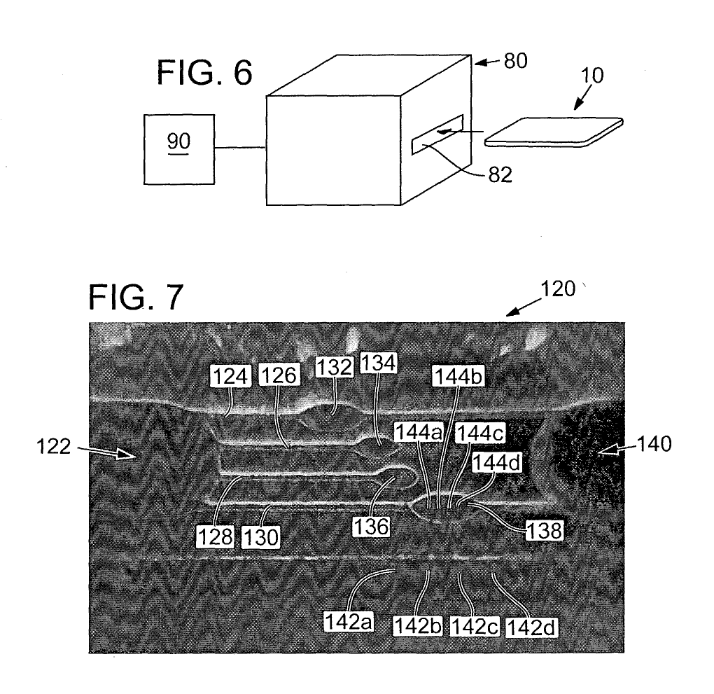

Lower board 14 defines an electrical chip 18 that provides the necessary electrical interconnects between selected reaction chambers in upper board 12 and the separate analytical instrument 80 shown in FIG. 6. With specific reference to FIGS. 2 and 4, reaction chambers 40 and 42 are configured to be electrical reaction chambers that are capable of testing a water sample for attributes that may be characterized by electrical properties of the sample. Reaction chamber 40 includes a four-terminal electrical circuit interface having four electrical traces 46a, 46b, 46c, and 46d that define probes that extend into reaction chamber 40 and make contact with a water sample contained in the reaction chamber. Each of the traces 46 has a bond pad 48 (48a, 48b, 48c and 48d) on the opposite end in a position on board 14 such that the bond pads 48 may be interconnected with a corresponding probe in the analytical instrument 80. The electrical reaction chambers 40, 42 may alternately be configured with a two-terminal electrical circuit rather than the four-terminal circuit just described. For example, reaction chamber 42 includes two electrical traces 50a and 50b that terminate on one end in reaction chamber 42 and which interconnect on the opposite end to a bond pad 52a and 52b, respectively.

The manner of fabricating the water analysis chip 10 will now be detailed prior to an explanation of the manner of using the chip.

Upper board 12 and lower board 14 are separately manufactured before the two boards are bonded together. Both upper and lower boards may be manufactured from silicon materials or glass substrates such as soda lime or borofloat, although other similar materials including various plastics may be used. Regardless of the material used to fabricate upper board 12, the material is selected so that the board is optically transparent so that, as detailed below, light from a light source in an analytical instrument 80 may be transmitted through the board material so that the analytical instrument detects calorimetric changes that occur in the chemical reaction type reaction chambers and optical characteristics of light transmitted through sample contained in optical chambers. Beginning with upper board 12, the substrate material is first pre-cleaned to remove and eliminate surface contamination such as particulate matter, organic molecules and metal traces. Then, using a photo patterning tool, the microfluidic fluid-carrying channels (i.e. 30, 32, 34 and 54, 56 and 58) and the reaction chambers (i.e. 36, 38, 40 and 42) and reference cell 60 are photo patterned onto the lower surface 26 of the board 12. The exposed portions of the lower surface are then etched according to, for example, a wet etch or plasma dry etch process. As an example of a wet etch process, a buffered oxide etch may be utilized. The depth of the fluid-carrying channels and of the reaction chambers is controlled through the etching process to achieve the desired dimensions and such that desired optical characteristics of light transmitted through the chip 10 are achieved. In the preferred embodiment, the reaction chambers and the fluid-carrying channels are of the same depth, and typical depths are from about 30 μm to about 100 μm, although these parameters may be varied widely according to need. It will be appreciated that the reaction chambers may be formed to be proportionately “deeper”-than the channels, that is, so that they extend further into the upper board 12 measured from lower surface 26 of the board than the channels. Once the channels and reaction chambers are formed, resist is stripped away from lower surface 26 and fluid inlet port 20 and air management port 22 are formed, for example by drilling the wafer substrate with a laser drill or other appropriate tool.

As noted, reaction chamber 36 is configured for performing chemical reaction-based analyses that result in calorimetric changes that are detected by the analytical instrument 80. To facilitate the desired chemical reactions in the chambers, different reagents and dyes and the like are deposited into the reaction chambers after the etching process. After a water sample is introduced into the reaction chamber, the reagent reacts with the water and produces calorimetric changes that are detected by the analytical instrument. The specific reagent and or reagents deposited in any given reaction chamber may be different from the reagents deposited in the adjacent reaction chamber. It will be appreciated, therefore, that any given chip 10 may include reaction chambers configured for carrying out any number of analyses. Thus, and by way of example only, and with reference to FIG. 2, reaction chamber 36 may include reagents appropriate for measuring free chlorine in a water sample. Reaction chamber 38 is as noted an optical chamber and thus does not include any reagents. For purposes of illustration, it will be assumed that chamber 38 is used for determining turbidity of a sample in the chamber. With a chip that includes a greater number of chemical reaction type chambers, other reagents specific to testing other water properties may be used. In practice, there are several chemical compounds that must be combined in order to test for chemical characteristics such as free chlorine. These compounds are combined in the reaction chambers, although they are referred to herein simply as a reagent.

It is often advantageous to deposit a matrix compound in the reaction chamber for the purpose of either physically entrapping or chemically binding the reagents, thereby maintaining the reagents in the reaction chamber prior to the time when a sample is introduced. There are many suitable matrix compounds that may be used for this purpose. For example, polyvinyl alcohol (PVA) deposited on the interior surface of the reaction chambers forms a physical matrix structure that is capable of entrapping various reagents. Other, sorbant-type materials may similarly be used to attract or bind both organic and inorganic reagent compounds, and may be combined with matrix compounds for binding reagents. Suitable sorbants include the classes of chemical sorbants commonly used in chromatographic columns. There are a wide variety of such sorbants available on the commercial market, and the specific type of sorbants selected depends upon numerous factors, including the type of test that is being run and the reagents used in the test, the size of the molecules involved, polarity, solubility, the environmental operating conditions, etc. Sorbants such as cross-linked cellulose or agarose, adsorbents used in liquid chromatography, and sorbants of the types often used in thin board chromatography may be used. Preferably, any matrix compounds and sorbant materials that are used are capable of being easily coated onto the walls of the reaction chambers, for example by applying a monolayer of the materials with techniques such as low volume fluid dispensing.

Turning now to the method of manufacturing lower board 14, the substrate material (which is preferably the same as the substrate material used to fabricate upper board 12, but which may in some instances be opaque rather than transparent) is pre-cleaned as described above with reference to board 12. The lower board 14 serves as the electrical test components of the chip 10, and also interfaces the chip with an analytical instrument 80. As such, the electrical traces and bond pads used in lower board 14 are designed so that they are positioned correctly when the two boards are assembled. Specifically, the traces (such as traces 46) are located in a position on the upper surface 70 of lower board 14 that the traces will terminate in reaction chamber 40 in upper board 12 when the two boards are bonded together. Likewise, the bond pads 48 are positioned in a position on the upper surface 70 of the lower board 14 that is near one side edge of the board. Assuming for purposes herein that silicon is used as the starting wafer substrate material for board 14, a thin oxide film is grown on the upper surface 70 of board 14. A metal film is then deposited by sputter coating on the upper surface 70. The specific type of metal film depends upon the type of electrical measurement that will be made in any given reaction chamber. For example, if the test that will be run is conductivity of the water sample, a low resistance metal film such as a tantalum (Ta)/gold (Au) film is preferred. This type of film is deposited by first depositing a thin layer of Ta to act as an adhesion layer between the Au and the wafer surface. The thickness of the Ta layer may be varied according to desired properties, and preferably is between a few Angstroms and several thousand Angstroms. Au is then deposited on top of the Ta. The thickness of the Au may be varied according to the circuitry needs and the electrical measurement characteristics required. Typically, the Au is deposited in a thickness between about 0.2 μm and about 1.5 μm. Either wet and plasma dry etching of the metal, or a combination of both, is next used to etch the desired pattern, after which remaining photo resist is stripped off the surface of the wafer.

A thin reflective film, the purpose of which is described in greater detail below, may be deposited on a surface of one of the boards, such as upper surface 70 of board 14 if desired. The reflective film assists in scattering light from analytical instrument 80 that is transmitted onto chip 10 during analytical analysis.

As noted, in some instances board 14 may be fabricated from an opaque material that is not optically transparent. In these instances the upper board must be fabricated from an optically transparent material.

With water analysis chip 16 and electrical chip 18 manufactured as described, the two chips are singulated and bonded to one another. Singulation refers to the process of forming a chip into a desired geometric configuration. In the instant case, each board 12 and 14 is first laminated onto a support structure. The boards and the associated support structures are then cut to the desired size and shape.

The two boards 12 and 14 are then oriented in a face-to-face manner—that is, with upper surface 70 of board 14 facing lower surface 26 of board 12, and with the electrical traces (e.g. 46, 50) oriented relative to the associated reaction chambers (e.g. 40, 42) that the traces will extend into the reaction chambers when the two boards are bonded together. The boards are bonded together in this desired orientation. The boards may be bonded together in any appropriate manner, for example with non-water soluble adhesives, thermal compression, or a polyamide and/or thermoset film. The bond pads 48, 52 are kept out of the interface between the two boards during bonding so that electrical probes in the analytical instrument 80 may establish electrical connections with the bond pads.

Water analysis chip 10 is used by introducing a sample of water into fluid inlet port 20. The water sample may be introduced into the inlet port in any convenient manner, such as with a dropper or pipette, with an injection needle, or for example by immersing the chip itself into a water sample so that the fluid inlet port is below the surface of the water. It should be noted that fluid inlet port 20 may be replaced with other equivalent structures for routing a water sample into the chip 10, including for example injection needles and the like. In any case, the water sample flows through inlet port 20 and is drawn through channels 30, 32 and 34 and into associated reaction chambers by passive capillarity—that is, the water sample flows into the reaction chambers without the need for an active mechanism for inducing fluid flow. Air that is displaced from the channels 30, 32 and 34 and associated reaction chambers by the fluid is ported through the air management port 22, which facilitates capillary flow. When glass is used to form the boards 12 and 14 and is sufficiently clean, the capillarity of the channels has been found to be sufficient. Nonetheless, the inlet port 20 and the microfluidic channels may optionally be treated with coatings or surface modification methods to assist in capillarity by, for example, preventing a meniscus from forming in the inlet port. The specific type of surface treatment depends upon the material used to manufacture the board 12. For example, some materials such as certain glasses may be cleaned according to SC1 clean techniques. In other cases, such as with various plastics, monolayers of surfactant compounds may be applied to the board. The air management port 22, as noted, facilitates the capillary flow of water through the channels and into the reaction chambers and ensures that the water sample flows into each reaction chamber, by allowing air displaced by the water sample as it moves through the microfluidic channels to be released through the port 22. Again, the function of air management port 22, which in the illustrated embodiment is ported to the atmosphere, may be equivalently performed by a closed air management chamber fluidly connected to the reaction chambers.

When a water sample enters reaction chamber 36 the reagents contained in the reaction chamber intermix and reacts with the water. The reagents are designed to generate a colorimetric change as the reaction occurs, and the change is detectable by the analytical instrument 80, as described below. The analytical instrument 80 also includes electrical probes that make an electrical connection with bond pads 48 and 52 to facilitate electrical tests on the water sample contained in reaction chambers 40 and 42.

With reference now to FIG. 6, an analytical instrument 80 is configured for running analytical tests on a water sample contained in a water analysis chip 10 that may be inserted into an analysis port 82 in the instrument. Analytical instrument 80 is shown and described in a general manner herein to provide some context for an analytical instrument used with chip 10. Analytical instrument 80 includes optical components suited for detecting colorimetric changes in a sample held in reaction chamber 36, for measuring optical properties of a sample held in optical chamber 38, electrical components for running electrical analyses with respect to samples held in reaction chambers 40, 42, for analyzing those optical and electrical data, and reporting the results of the analysis in the form of data that may be saved in internal memory in analytical instrument 80, and/or output to a computer 90. In a preferred embodiment, analytical instrument 80 is a self-contained unit that is easily transported into the field, and computer 90 is a portable unit such as a handheld or laptop computer.

When a water sample is introduced into the water analysis chip 10 and the associated reaction chambers, the chip 10 is allowed sufficient time for chemical reactions to take place in the reaction chambers. Again, the analytical test that is run in any given reaction chamber will vary according to need, and according to the reagents that are contained in the reaction chamber. Following the example given above, and for purposes of explanation, reaction chamber 36 will be assumed to include reagents appropriate for measuring free chlorine in the water sample contained in that reaction chamber. Reaction chamber 38 is an optical chamber and thus includes no reagents, but is intended for measurement of turbidity. The reactions that occur in reaction chamber 36, and the properties of sample contained in chamber 38, are detectable by the optical character of light that is either transmitted through the water analysis chip 10, or in the instance where a reflective film is applied to a surface such as surface 70, light that is transmitted through the water sample and reflected from the reflective film to an appropriate detector.

As noted, in some instances a thin reflective film may be applied to a surface of one of the boards, for example upper surface 24 of upper board 12, or the lower surface of lower board 14, and the like. The reflective film is preferably a white film that serves to optically scatter light from the light source in analytical instrument 80, but which also may be a reflective film such as aluminum. When this type of construction is used, light from the light source in analytical instrument 80 is reflected off the reflective film and is transmitted to the detector.

Analytical instrument 80 also includes electrical interconnects that establish an electrical connection between the analytical instrument 80 and its associated processors and bond pads 48 and 50 on chip 10.

The analytical steps performed in analytical instrument 80 will now be briefly explained with reference to two different analytical methods. According to the first method, with water analysis chip 10 containing a water sample and having had sufficient time for the chemical reaction to complete in the reaction chamber 36, the chip 10 is inserted into analytical instrument 80 via port 82 (as shown in FIG. 6), and light having the desired optical characteristics such as intensity and wavelength is transmitted with an analytical light source contained in the instrument through the reaction chambers in chip 10. The optical characteristics of the transmitted light is then analyzed by processors in the analytical instrument, which includes processors preprogrammed with algorithms to process the data from the light transmitted through the reaction chambers to measure free chlorine (in the instance of data from reaction chamber 36). Likewise, light transmitted through sample contained in optical chamber 38 is processed and the data is correlated to a measurement of turbidity. The optical characteristics of light transmitted through the sample contained in reaction chambers 36 and 38 is correlated to the chemical or physical property being measured—free chlorine in reaction chamber 36 and turbidity in optical chamber 38. Light transmitted through reference cell 60 is used as a control value for standardization purposes.

According to the second method, the water analysis chip 10 is inserted into analytical instrument 80 via port 82 (as shown in FIG. 6) immediately after a water sample is introduced into the chip. Light having the desired optical characteristics such as intensity and wavelength is transmitted with an analytical light source contained in the instrument through chip 10 on either a continuous or predetermined intermittent basis. The optical characteristics of the transmitted light is then analyzed by the processors in the analytical instrument over time, and the analysis continues (either continuously or intermittently) until the signal stabilizes—that is, until the reaction in the reaction chamber or optical chamber is complete. Reaction time is dependent upon the parameter being tested, and can vary from a few seconds to a few minutes. The data generated according to this method is processed to measure, for example, free chlorine (in the instance of data from reaction chamber 36). Likewise, light transmitted through a sample contained in optical chamber 38 is processed and the data is correlated to a measurement of turbidity.

The operational steps described above may be illustrated with reference to FIG. 9. A sample of water to be analyzed is first obtained as shown by 102. The sample may be acquired in any suitable manner, as detailed above, and is then introduced into at 104 into chip 10 and the sample flows by capillary action into the reaction chamber where the reactions take place (106). The “reactions” illustrated at 106 in FIG. 9 may be of the chemical type, electrical and/or optical types. The chip 10 is then inserted into analytical instrument 80 for analysis at block 108. Data from analysis 108 is output as described above and is collected at data collection 110.

Regardless of which method described above is used, analytical instrument 80 also sends appropriately conditioned electric signals to reaction chambers 40, 42 via bond pads 48 and 52 and the associated electrical traces 50, 46. These signals are processed into data associated with electrical analyses such as conductivity and temperature of the water sample contained in these reaction chambers.

Data from analytical instrument 80 may be output to computer 90, or saved in memory in instrument 80 (not shown). The analytical instrument 80 may be programmed with instructions of varying complexity, depending upon the specific needs of the situation.

Turning now to FIG. 7, a portion of a water analysis chip 120 is shown in a photomicrograph. In the embodiment illustrated in this photomicrograph, a water sample reservoir 122 is fluidly connected via four separate capillary channels 124, 126, 128 and 130 to four separate reaction chambers 132, 134, 136 and 138. Reaction chambers 132 and 138 are fluidly connected to an air management reservoir 140 through capillary channels 124 and 130, respectively, but reaction chambers 134 and 136 are not fluidly connected to an air management chamber of any type. The embodiment of FIG. 7 thus illustrates that an air management chamber or reservoir is optional, and that a water sample may be transported into a dead-end reaction chamber such as 134 and 136 through capillary movement without additional porting for the chambers. Three of the reaction chambers shown in FIG. 7 are of either the chemical reaction type that contain reagents, and are thus configured for running tests that are measured via calorimetric changes, or of the optical chamber type that are configured for running tests based solely on the optical characteristics of the sample contained therein—chambers 132, 134 and 136. Chamber 138 on the other hand is an electrical reaction chamber suited to such tests as conductivity of a sample contained therein, and is provided with a four terminal test circuit as shown with bond pads 142a, 142b, 142c and 142d, and the associated electrical traces 144a, 144b, 144c and 144d.

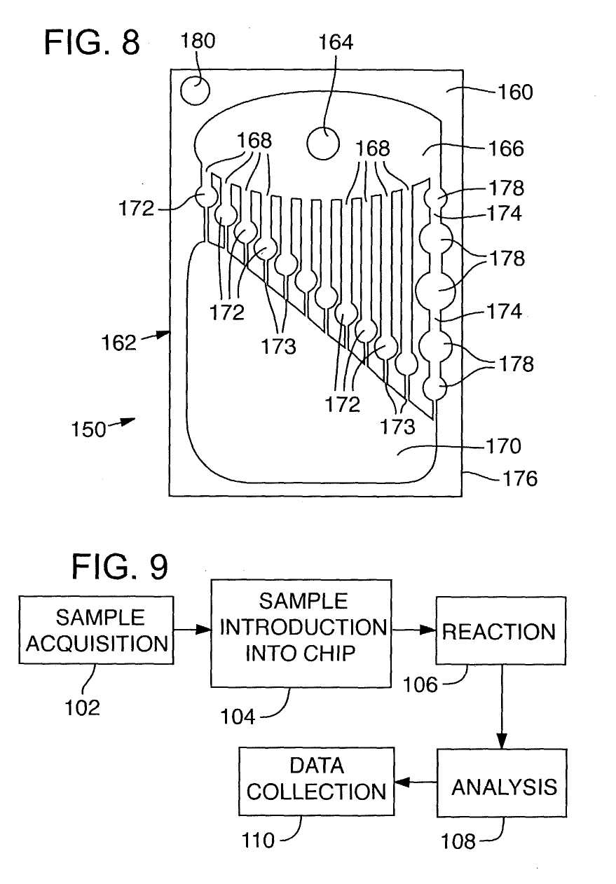

FIG. 8 illustrates yet another embodiment of a water analysis chip 150 according to the illustrated invention, illustrating only the lower surface 160 of the upper board 162 of the chip. In the embodiment shown in FIG. 8 the upper board 162 contains various fluid ports, channels and reaction chambers, similar to water analysis chip 12 described above. A fluid sample entry port 164 communicates through the board 162 to a sample reservoir 166 and provides an opening through which water samples are routed into the chip. Each of a plurality of microfluidic channels 168 communicates with a separate reaction chamber 172 that is defined along the length of each of the microfluidic channels 168. Relatively smaller microfluidic channels 173 extend between the reaction chambers 172 and a relatively large air management chamber 170 that is not ported to the atmosphere. Reaction chambers 172 are of the chemical reaction types that include reagents (bound or contained therein in the manner described above) specific to predetermined chemical analysis of a water sample introduced into the chambers, or the optical chamber type. A microfluidic channel 174 is located along one lateral edge 176 of chip 150 and has plural electrical type reaction chambers 178 located along the length of the channel. Reaction chambers 178 are of the types that communicate with electric terminals formed on the lower board (not illustrated in FIG. 8) that will be bonded to upper board 162, as described above, to facilitate electric analysis of a water sample introduced into the chambers 178. Channel 174 communicates at one end with sample reservoir 166 and at the other end with air management reservoir 170.

The embodiment of FIG. 8 is manufactured in the same manner as described above with respect to the embodiment of FIG. 1, but illustrates just one of the many forms that the water analysis chip 150 may take. The lower board (not shown) defines the electrical chip. As noted, the air management reservoir 170 of chip 150 does not communicate through the chip to the external atmosphere, and the channels 173 are smaller than the channels 168. Water will flow through the channels 168, but the channels 173 are small enough that water will not enter them from the reaction chambers 172. Air displaced by the water as it moves through the channels 168 and into the reaction chambers 172 will, however, move through the channels 173 and into the air management reservoir 170. Water however will not flow into channels 173 because those channels are too small for water to enter. It will thus be appreciated that the volume of the void defined by the air management reservoir may be varied to control the capillarity of the microfluidic channels 168. Chip 150 also includes a reference cell 180 for the purposes previously described.

Having here described illustrated embodiments of the invention, it is anticipated that other modifications may be made thereto within the scope of the invention by those of ordinary skill in the art. It will thus be appreciated and understood that the spirit and scope of the invention is not limited to those embodiments, but extend to the various modifications and equivalents as defined in the appended claims.

What is claimed is:

1. A chip for use in water analysis comprising a member defining a water inlet, at least one water carrying microfluidic channel fluidly connected within the member to the water inlet, at least one reaction chamber fluidly connected to the at least one water carrying microfluidic channel, and at least one air management chamber fluidly connected to the reaction chamber.

2. The water analysis chip according to claim 1 in which the reaction chamber includes a reagent deposited therein configured for testing a water sample for a predetermined chemical characteristic.

3. The water analysis chip according to claim 2 wherein the reagent is deposited into the reaction chamber during manufacture of the water analysis chip.

4. The water analysis chip according to claim 3 wherein the reagent is held in the reaction chamber in a surface coating deposited in the reaction chamber, and wherein the surface coating defines a physical matrix structure that entraps the reagent.

5. The water analysis chip according to claim 1 in which the reaction chamber includes an electrical circuit for testing a water sample for a predetermined electrical characteristic.

6. The water analysis chip according to claim 1 wherein the air management chamber defines air management means for enhancing passive capillarity of the at least one water carrying channel.

7. The water analysis chip according to claim 1 including plural water carrying microfluidic channels, each fluidly connected to the water inlet, each fluidly connected to a reaction chamber, and each fluidly connected to an air management chamber.

8. The water analysis chip according to claim 7 including plural reaction chambers that each include a reagent deposited therein configured for testing a water sample for a predetermined chemical characteristic.

9. The water analysis chip according to claim 7 including plural chambers that each include an electrical circuit for testing a water sample for a predetermined electrical characteristic.

10. The water analysis chip according to claim 9 including plural chambers that each include electrical probes extending into the reaction chambers for testing a water sample for a predetermined electrical characteristic.

11. The water analysis chip according to claim 8 wherein each reaction chamber is fluidly connected to an air management chamber.

12. The water analysis chip according to claim 1 in which the air management chamber is open to the atmosphere.

13. The water analysis chip according to claim 1 in which the air management chamber is defined by a chamber that is not open to the atmosphere.

14. The water analysis chip according to claim 1 wherein the member is optically transparent and further comprises a composite structure defined by an upper board and a lower board, each having an upper surface and a lower surface, the upper board has an opening from the upper surface to the lower surface defining the water inlet, and the at least one channel and reaction chamber are formed in the lower surface of the upper board.

15. The water analysis chip according to claim 14 wherein the air management chamber comprises an opening extending from the upper surface to the lower surface of the upper board.

16. The water analysis chip according to claim 1 further including a reference chamber defined by a void in the member.

17. The water analysis chip according to claim 1 wherein at least one reaction chamber includes electrical probes therein for testing desired electrical attributes of a sample of water contained in the reaction chamber.

18. The water analysis chip according to claim 1 including at least one reaction chamber having a reagent therein for reacting with a sample of water contained in the reaction chamber to test the water for a predetermined chemical attribute, at least one reaction chamber having electrical probes therein for testing desired electrical attributes of a sample of water contained in the reaction chamber, and at least one reaction chamber for testing an optical characteristic of a sample of water contained therein.

19. A method of analyzing water for predetermined chemical or physical attributes, comprising the steps of:
(a) introducing water into an inlet in an optically transparent water analysis chip,
(b) inducing a flow of the water by passive capillarity from the inlet through a fluid pathway and into a reaction chamber, wherein the reaction chamber is fluidly connected to an air management chamber;
(c) transmitting light having desired optical characteristics through the water in the reaction chamber;
(d) analyzing the light transmitted through the water in the reaction chamber.

20. The method according to claim 19 including the step of fixing reagents in the reaction chamber prior to introduction of water into the reaction chamber, and wherein the method includes the step of analyzing the water for a chemical attribute.

21. The method according to claim 19 including the step of providing electrical probes in the reaction chamber and exposing the water to the electrical probes, and wherein the method includes the step of analyzing the water for an electrical attribute.

22. The method according to claim 19 wherein the method includes the step of analyzing the water for an optical attribute.

23. A chip for use in fluid sample analysis, comprising:
a substrate defining a fluid inlet, a microfluidic fluid carrying channel within the substrate connected to the inlet, a reaction chamber within the substrate and connected to microfluidic fluid carrying channel, and an air management chamber fluidly connected to the reaction chamber to facilitated capillary flow of a fluid from the fluid inlet to the reaction chamber.

24. The fluid sample analysis chip according to claim 23 wherein the reaction chamber further comprises first reaction chamber for testing a sample contained in the first reaction chamber for a predetermined chemical characteristic, a second reaction chamber for testing a sample contained in the second reaction chamber for an electrical characteristic, and a third reaction chamber for testing a sample contained in the third reaction chamber for an optical characteristic.

25. The fluid sample analysis chip according to claim 23 including a reagent deposited in the reaction chamber, the reagent configured for testing a fluid sample contained in the reaction chamber for a predetermined chemical characteristic.

26. The fluid sample analysis chip according to claim 25 wherein the reagent is loaded into the reaction chamber during manufacture of the water analysis chip.

27. The fluid sample analysis chip according to claim 24 wherein the air management chamber defines means for facilitating capillary flow of fluid from the inlet to the reaction chamber.

28. A method of making a water analysis chip comprising the steps of:
forming in a substrate a water inlet, an internal microfluidic channel connected to the inlet, an internal reaction chamber connected to the microfluidic channel and an air management chamber connected to the reaction chamber.

29. The method according to claim 28 including the step of depositing in the reaction chamber a reagent configured for testing a water sample for a predetermined chemical characteristic.

30. The method according to claim 28 including the step of forming plural reaction chambers wherein at least one of the reaction chambers includes electrical probes therein.

31. The method according to claim 30 further comprising:
(a) forming in a first board having opposed surfaces the water inlet such that the inlet defines an opening through both surfaces;
(b) forming the microfluidic channel and reaction chamber in one surface; and
(c) bonding the first board to a second board in a desired spatial relationship such that the first and second boards when bonded define the chip, and wherein at least one of the first or second boards is optically transparent.

32. The method according to claim 31 further including the step of providing on the second board electrical probes having a first end extending into at least one reaction chamber when the first and second boards are bonded.

33. A water analysis chip, comprising:
a substrate member having
an inlet port;
at least one microfluidic channel connected to the inlet port;
at least one reaction chamber means connected to the microfluidic channel for performing analytical analyses of a sample of water in the reaction chamber means, and
air management means connected to the reaction chamber for facilitating capillary flow of fluid from the inlet to the reaction chamber; and
wherein said substrate member is at least partly optically transparent so that light may be transmitted from an external light source through the reaction chamber means.

34. The water analysis chip according to claim 33 wherein the reaction chamber means further comprises an internal void in the substrate member having a reagent deposited therein, wherein the reagent is configured for reacting with a sample of water contained in the reaction chamber to test the sample for a predetermined chemical characteristic.

35. The water analysis chip according to claim 33 wherein the reaction chamber means further comprises an internal void in the substrate member having electrical interconnects in contact with the void, the electrical interconnects configured to test a sample of water contained in the reaction chamber for a predetermined electrical characteristic.

36. The water analysis chip according to claim 33 wherein the reaction chamber means further comprises an internal void in the substrate member configured to test a sample of water contained in the reaction chamber for a predetermined optical characteristic.

37. The water analysis chip according to claim 33 wherein the air management means comprises an outlet port opening to atmosphere.

38. The water analysis chip according to claim 33 wherein the air management means comprises an internal void in the substrate member closed to atmosphere.

39. The water analysis chip according to claim 34 including plural reaction chamber means, each having a different reagent deposited therein for reacting with a water sample contained in the respective plural reaction chambers to test the sample for a different predetermined chemical characteristic.
